(19) United States
(12) Patent Application Publication
(10) Pub. No.: US 2008/0031905 A1
(43) Pub. Date: Feb. 7, 2008

(54) METHOD FOR TREATMENT OF BURNS

(76) Inventors: John O. Cram, Hilton Head Island, SC (US); Miguel Castillo, Hilton Head Island, SC (US)

Correspondence Address: WILLIAM H. EILBERG, THREE BALA PLAZA, SUITE 501 WEST, BALA CYNWYD, PA, 19004, US

(21) Appl. No.: 11/461,796

(22) Filed: Aug. 2, 2006

(51) Int. Cl. A61K 31/22

(52) U.S. Cl. 424/401; 514/547

(57) ABSTRACT

A method of treating minor burns includes applying a water-based wax emulsion to the area of the burn. When the emulsion dries, it forms a thin, protective film over the burn. Application of the emulsion has been found to provide temporary and immediate relief of pain and/or itching. The protective film shields the burn, and therefore aids in healing. The emulsion can be made of any of a variety of waxes, and the emulsion can be formed with long-chain alcohol or fatty acid soaps, such as oleic acid or triethanolamine, or any other materials that form a water-wax emulsion. The emulsion may be combined with a coloring agent and/or a perfume which will not affect the function of the emulsion.

### BACKGROUND OF THE INVENTION

The present invention is a method of treating minor burns, and provides temporary relief of pain and itching, while promoting healing.

Copending U.S. patent application Ser. No. 11/203,697, the disclosure of which is incorporated by reference herein, describes a composition and method for treatment of insect bites and the like. In brief, the method described in the cited application includes applying a water-based wax emulsion to the site of a wound.

The present invention results from the discovery that the method described in the above-cited patent application is also effective for treatment of minor burns. As in the cited application, the water-based wax emulsion used in the present invention has been found to work well in relieving pain and itching, and in promoting healing.

### SUMMARY OF THE INVENTION

The present invention comprises treating a minor burn with a water-based wax emulsion.

The water-based wax emulsion is applied over the burn. When the emulsion dries, it forms a thin protective film over the burn. Application of the emulsion has been found to provide temporary and immediate relief of pain and/or itching. The protective film shields the area of the burn, and therefore aids in healing.

The water-based wax emulsion used in the present invention can be made of any of a variety of types of wax, such as carnauba wax, paraffin, microcrystalline wax, bees wax, montan wax, candelilla wax, shellac, or other waxes, or any combination of the foregoing. The material used to form the emulsion may be long-chain alcohol or fatty acid soaps, and may include substances such as oleic acid or triethanolamine, or any other materials that form a stable water/wax emulsion.

The water-based wax emulsion may also include a coloring agent, and/or a perfume, which will not affect the function of the wax emulsion.

The water-based wax emulsion used in the present invention does not contain any organic solvents or flammable liquids. The use of a non-flammable wax emulsion makes it practical to store the material conveniently in virtually any container, including a plastic container.

The present invention therefore has the primary object of providing a method of treating minor burns.

The invention has the further object of providing a material for treatment of burns, which material can be safely and conveniently stored in plastic containers.

The invention has the further object of providing a method of treating minor burns, wherein the method provides temporary but immediate relief of pain and/or itching, and wherein the method also promotes healing of the burn.

The reader skilled in the art will recognize other objects and advantages of the present invention, from a reading of the following detailed description of the invention, and the appended claims.

### DETAILED DESCRIPTION OF THE INVENTION

The present invention comprises the treatment of minor burns with a water-based wax emulsion. It has been found that the wax emulsion can be used as a skin medication to treat such burns. The emulsion dries to form a substantially waterproof, thin, protective film over the area of the burn. The emulsion thus promotes healing of the burn.

The wax emulsion, when applied to the site of a burn, has been found, upon drying, to soothe the pain caused by the burn. The product forms a protective film over the burn, effectively encapsulating the burn. Application of the wax emulsion tends to result in a smaller blister than would appear in the absence of such treatment. It is believed that the smaller blister is less likely to push against nerve endings, thereby reducing the pain experienced.

The wax emulsion can be made from many possible types of wax. These types include carnauba wax, paraffin, microcrystalline wax, bees wax, montan wax, candelilla wax, shellac, or similar types of wax, or any combination of the foregoing.

The above-described waxes, which are insoluble in water, are emulsified with long-chain alcohol or fatty acid soaps, which reduce the surface tension at the interface of the suspended wax particles because of the solubility properties of their molecules.

The materials used to form the emulsion could be, for example, oleic acid or triethanolamine, or any other materials that form a water/wax emulsion. This allows the waxes to remain in a stable solution or emulsion. The invention is not limited to the specific examples given above.

The water-based wax emulsion may be combined with a coloring agent for purposes of identification. The coloring agent is preferably a water-based dye or food coloring agent. The coloring agent should be chosen such that it does not interfere with the function of the emulsion. Subject to the above limitation, any suitable dye can be used.

The water-based wax emulsion can further be combined with a perfume, or the like, to make the product more pleasant for use by the consumer. Any suitable perfume could be used.

In one example, a dye used to color the water-based wax emulsion could be selected from the group consisting of tetraethylrhodamine, D & C Red No. 19, rhodamine B chloride, C.I. Basic Violet, and C.I. 45170. The chemical designation for a particular dye which has been used successfully with the present invention is N-[9-(2-carboxyphenyl)-6-(diethylamino)-3H-xanthen-3-ylidene]-N-ethylethanaminium chloride. The invention is not limited to use with the specific items listed above.

When the water-based wax emulsion is applied to the area of the burn, and when the water in the emulsion evaporates, the wax or waxes are left in a coherent water-resistant smooth film which covers the burn. This film prevents or stops itching, protects the burn, and therefore aids in healing.

Because the present invention uses inert waxes and a soap-based emulsification system, which materials will not readily react with other materials, including container materials, the compositions used in the present invention can conveniently be stored in plastic containers or bottles.

While wax emulsions have been known in the prior art, it has not been known to use such materials for treating minor burns.

The wax emulsion used in the present invention may be packaged in a small bottle, containing one or two fluid ounces, with an applicator similar to what is used with a bottle of mercurochrome or iodine. The material can be packed in almost any type of plastic bottle. Unlike the case of some organic solvents, it is not necessary to store the wax emulsion in a glass container.

The invention can be modified in various ways. The wax used in making the emulsion is not limited to the materials listed above, but can include other materials not specifically mentioned. The substances used to form the emulsion can also be varied. Different coloring agents can be used, and the invention is not limited to use with a specific perfume. These and other modifications, which will be apparent to the reader skilled in the art, should be considered within the spirit and scope of the following claims.

What is claimed is:

1. A method of treating a minor burn, comprising applying, to a site of a burn, a water-based wax emulsion.

2. The method of claim 1, further comprising the step of allowing the emulsion to dry, so as to form a substantially waterproof, thin film.

3. The method of claim 1, wherein the emulsion is selected to contain a wax which is selected from the group consisting of carnauba wax, paraffin, microcrystalline wax, bees wax, montan wax, candelilla wax, shellac, or any combination of the foregoing.

4. The method of claim 3, wherein the emulsion is combined with a coloring agent.

5. The method of claim 3, wherein the emulsion is combined with a perfume.

6. The method of claim 1, wherein the emulsion is formed with a long-chain alcohol or a fatty acid soap.

7. The method of claim 3, wherein the emulsion is formed with a long-chain alcohol or a fatty acid soap.

8. The method of claim 1, wherein the emulsion is formed with a material selected from the group consisting of oleic acid and triethanolamine.

9. The method of claim 1, further comprising the step of storing the water-based wax emulsion in a plastic container.

10. A method of treating a burn, comprising applying, to a site of a burn, a water-based wax emulsion, wherein the emulsion is selected to contain a wax which is selected from the group consisting of carnauba wax, paraffin, microcrystalline wax, bees wax, montan wax, candelilla wax, shellac, or any combination of the foregoing, and allowing the emulsion to dry, so as to form a substantially waterproof, thin film.

11. The method of claim 10, wherein the emulsion is formed with a material selected from the group consisting of long-chain alcohols and fatty acid soaps.

12. The method of claim 10, wherein the emulsion is formed with a material selected from the group consisting of oleic acid and triethanolamine.

13. The method of claim 10, further comprising the step of storing the water-based wax emulsion in a plastic container.

14. A method of treating a minor burn, comprising forming a water-based wax emulsion, the emulsion containing wax selected from the group consisting of carnauba wax, paraffin, microcrystalline wax, bees wax, montan wax, candelilla wax, shellac, or similar types of wax, or any combination of the foregoing, applying the emulsion to a burn, and allowing the emulsion to dry so as to provide a protective film over the burn.

15. The method of claim 14, wherein the emulsion is selected to include a material selected from the group consisting of long-chain alcohols and fatty acid soaps.

16. The method of claim 14, wherein the emulsion is selected to contain a material selected from the group consisting of oleic acid and triethanolamine.

17. The method of claim 14, further comprising the step of storing the water-based wax emulsion in a plastic container.

18. The method of claim 14, further comprising combining the water-based wax emulsion with a coloring agent before applying the emulsion to the burn.

19. The method of claim 14, further comprising combining the water-based wax emulsion with a perfume before applying the emulsion to the burn.

20. The method of claim 18, further comprising selecting the coloring agent such that the coloring agent does not interfere with a function of the water-based wax emulsion.
